(19) United States
(12) Patent Application Publication
(10) Pub. No.: US 2016/0095741 A1
(43) Pub. Date: Apr. 7, 2016

(54) BRUXISM GUARD WITH MANDIBLE COMPENSATION MECHANISM

(71) Applicants: 12th Man Technologies, Inc., Garden Grove, CA (US); DDME Inc., Las Vegas, NV (US)

(72) Inventors: Alex Stenzler, Long Beach, CA (US); William Ross, Las Vegas, NV (US); Jeffory Wyscarver, Corona, CA (US); Steve Han, Huntington Beach, CA (US)

(21) Appl. No.: 14/873,464

(22) Filed: Oct. 2, 2015

Related U.S. Application Data
(60) Provisional application No. 62/059,651, filed on Oct. 3, 2014.

(51) Int. Cl. A61F 5/56

(52) U.S. Cl. CPC A61F 5/566; A61F 2005/563

(57) ABSTRACT

A bruxism guard is described. The bruxism guard includes a first U shaped tray having a top surface and a bottom surface, the top surface including a trough with a dental insert positioned therein, wherein the dental insert substantially conforms to a full arch of a subject's teeth, and a second U shaped tray having a top surface and a bottom surface, the top surface including a trough with a dental insert positioned therein, wherein the dental insert substantially conforms to a only a partial arch of the subject's teeth, wherein the bottom surfaces of the first and second trays oppose each other when positioned within the subject's mouth. Also described is a bruxism guard having a removable T-Bar that permits the bruxism guard to be interlocked in at least four different configurations.

### CROSS-REFERENCE TO RELATED APPLICATIONS

This application claims priority to U.S. provisional application No. 62/059,651 filed on Oct. 3, 2014 incorporated herein by reference in its entirety.

### BACKGROUND OF THE INVENTION

Bruxism afflicts many people and is characterized by the unconscious grinding, gnashing, and clenching together of teeth. It may occur with enough frequency and severity to cause jaw disorders, headaches, damaged teeth and many other issues, including sleep apnea and snoring. Relief of bruxism is often sought from oral devices such as bite plates and splints.

For example, the most common oral device for treating bruxism is a single arch splint that is typically formed from hard plastic and fits over the entire row of either the upper or lower teeth (most commonly the upper teeth). Unfortunately, this type of structure crowds the oral cavity and displaces the tongue backwards when fitted to the user's teeth. It is well known that a shift in the tongue by mere millimeters may cause a repositioning in the soft tissues associated with the mandible, resulting in an increase in the resistance of the airways during breathing. Such a device also lacks any mechanism to compensate for the user's clenching forces, which triggers bursts of muscular activity in the masseter and other muscles associated with the movement of the jaw. As a result, a single arch occlusal splint is only able to protect the user's dentition by preventing contact between the upper and lower teeth, but doesn't address the problems of jaw pain, and actually exacerbates issues pertaining to sleep apnea and snoring due to the undesired repositioning of the tongue.

The top surfaces of the teeth have irregular surfaces and these cusps naturally interlock to some degree with this interference somewhat limiting the sliding of the two tooth surfaces when in contact. The application of either a single or double arch splint, covers these irregular surfaces and enables the lower jaw to slide back, increasing airway obstruction.

A second type of commonly used oral device is the anterior bite plane. This device only covers the anterior teeth of one arch and is a minimal presence in the oral cavity. By virtue of points of contact with only the anterior teeth, the upper and lower posterior teeth are separated by a space. This separation of posterior teeth helps to decrease muscle activity during bruxism. However, due to clenching forces being distributed only on the anterior teeth and no resistance on the anterior teeth, prolonged use may lead to intrusions of anterior teeth and supraeruption of the posterior teeth. Users may also experience joint pain since versions of the anterior bite plane do not allow for lateral movement of the jaw and hold the jaw in a single position for all hours of sleep. This device is also more difficult to use in people with a larger than normal overbite.

A third type of commonly used oral device is the full oral appliance that covers both the upper and lower arches of teeth. Such a device offers as much protection to the teeth as the single arch splint and is additionally able to reposition the lower jaw to relieve airway obstruction. However, due to its total coverage of the upper and lower arches, no space is allowed between posterior teeth and clenching forces may continue to trigger the firing of jaw muscles to lead to pain and jaw disorders.

Thus, there is need in the art for a device that is able to protect the teeth from bruxism, provides separation of teeth to offload jaw muscles, and enables positioning of the jaw to compensate for the displacement of the tongue while wearing the device and provide for some lateral and anterior/posterior movement of the jaw to prevent the jaw pain associated with its use. The present invention satisfies this need.

### SUMMARY OF THE INVENTION

The present invention relates to a bruxism guard that can maintain the jaw in a neutral or its natural position. The bruxism guard includes a first U shaped tray having a top surface and a bottom surface, the top surface including a trough with a dental insert positioned therein, wherein the dental insert substantially conforms to a full arch of a subject's teeth, and a second U shaped tray having a top surface and a bottom surface, the top surface including a trough with a dental insert positioned therein, wherein the dental insert substantially conforms to a only a partial arch of the subject's teeth, wherein the bottom surfaces of the first and second trays oppose each other when positioned within the subject's mouth. In one embodiment, the dental insert of the first tray conforms to the subject's mandibular dentition and the dental insert of the second tray conforms to the subject's maxillary dentition. In another embodiment, the bruxism guard includes an interlocking mechanism to temporarily secure the first tray to the second tray. In another embodiment, the interlocking mechanism permits limited forward and backward movement between the first and second trays. In another embodiment, the bottom surface of the first component includes a plurality of slots at its arcuate end, and the bottom surface of the second component includes a T-Bar sized to temporarily engage any one of the plurality of slots in the first component. In another embodiment, the slot nearest the far edge of the arcuate end of the first component is wider than a second adjacent slot. In another embodiment, at least one of the first or second trays includes a bite plate on the bottom surface of the tray. In another embodiment, the dental insert of the second tray cover's the only the incisors and canines of the subject's arch of teeth. In another embodiment, the dental insert of the second tray cover's the only the incisors, canines and first premolars of the subject's arch of teeth. In another embodiment, the dental insert of the second tray cover's the only the incisors, canines and premolars of the subject's arch of teeth. In another embodiment, the dental insert of the second tray cover's the only the incisors, canines, premolars and first molars of the subject's arch of teeth. In another embodiment, the dental insert of the second tray cover's the only the incisors, canines, premolars and first and second molars of the subject's arch of teeth.

The present invention also relates to a bruxism guard having a first U shaped tray with a top surface and a bottom surface, the top surface including a trough with an opening positioned therein, a T-Bar having a base plate, a post extending off center from the base plate and a flange at the extended end of the post, and a second U shaped tray having a top surface and a bottom surface, the top surface including a trough with at least one slot creating an opening therethrough, wherein when the T-Bar is positioned in the opening of the first tray with the T-Bar post and flange extending therethrough, the T-Bar temporarily engages one of the at least one slots to interlock the first tray to the second tray. In one embodiment, the first tray and the second tray are capable of interlocking in at least four different configurations. In another embodiment, the bruxism guard further includes a dental insert positioned within the trough of each of the first tray and the second tray. In another embodiment, at least one of the dental inserts substantially conforms to a full arch of the subject's teeth. In another embodiment, at least one of the dental inserts substantially conforms to a partial arch of the subject's teeth. In another embodiment, one dental insert substantially conforms to a partial arch of the subject's teeth and the second dental insert substantially conforms to a full arch of the subject's teeth. In another embodiment, slight forward and backward movement between the first and second trays is permitted when the T-Bar engages the at least one slot. In another embodiment, the second tray includes two or more slots, and the width of the first slot is greater than the width of the second slot.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1B, is a schematic of the top (1A) and bottom (1B) view of a first exemplary tray component of the present invention.

FIG. 2, comprising FIGS. 2A-2B, is a schematic of the top (2A) and bottom (2B) view of a second exemplary tray component of the present invention.

FIG. 3, comprising FIGS. 3A-3D, is an image of a first tray component (3A), a second tray component (3B), and dental inserts isolated (3A) and positioned within (3B) the exemplary tray components of the present invention.

FIG. 4, comprising FIGS. 4A-4B, is a side view (4A) and a front view (4B) of the exemplary trays and dental inserts assembled for positioning in a subject's mouth.

FIG. 5, comprising FIGS. 5A-5B, is a schematic of an alternative first tray component (5A) with an insertable T-Bar (5B).

FIG. 6, comprising FIGS. 6A-6B, is an exploded view schematic of the T-Bar, first tray and second tray components with the T-Bar aligned for insertion in a outward position (6A) or an inward position (6B).

FIG. 7, comprising FIGS. 7A-7B, is a schematic of the bruxism guard locked into a first position for the mandible being largely retracted. FIG. 7A is a bottom view, while FIG. 7B is a side view.

FIG. 8, comprising FIGS. 8A-8B, is a schematic of the bruxism guard locked into a second position for the mandible being slightly retracted. FIG. 8A is a bottom view, while FIG. 8B is a side view.

FIG. 9, comprising FIGS. 9A-9B, is a schematic of the bruxism guard locked into a third position for the mandible being slightly protracted. FIG. 9A is a bottom view, while FIG. 9B is a side view.

FIG. 10, comprising FIGS. 10A-10B, is a schematic of the bruxism guard locked into a third position for the mandible being largely protracted. FIG. 10A is a bottom view, while FIG. 10B is a side view.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical bruxism guards. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

In a first aspect, the present invention includes a device or assembly comprised of first and second interlockable teeth trays or components corresponding to the upper and lower arches, with dental inserts positioned therein. For example, one maxillary component and dental insert conforms to at least a portion of the upper maxillary arch of teeth and one mandibular component and dental insert conforms to at least a portion of the lower mandibular arch of teeth.

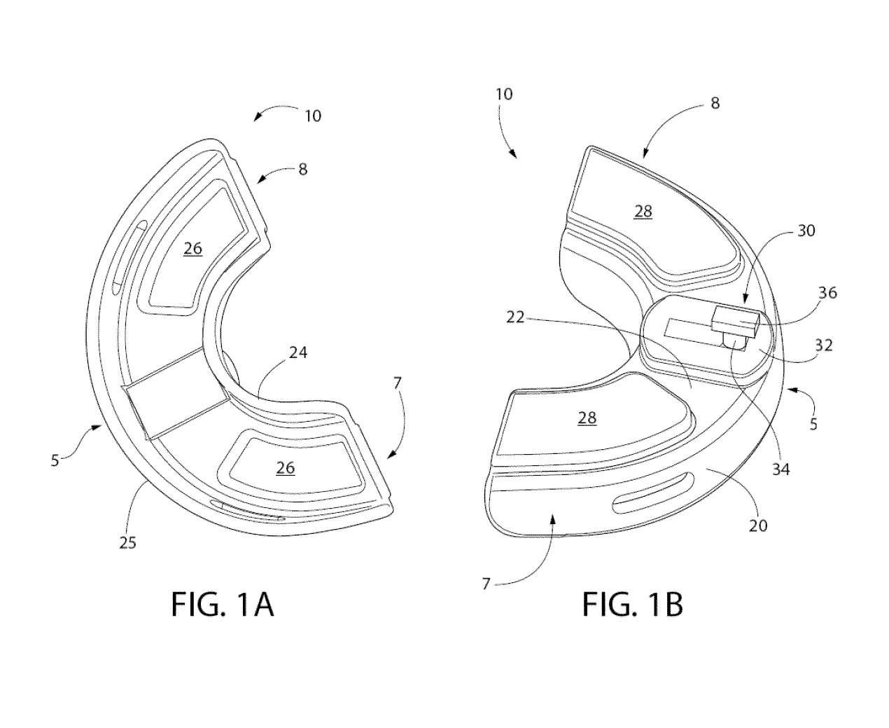

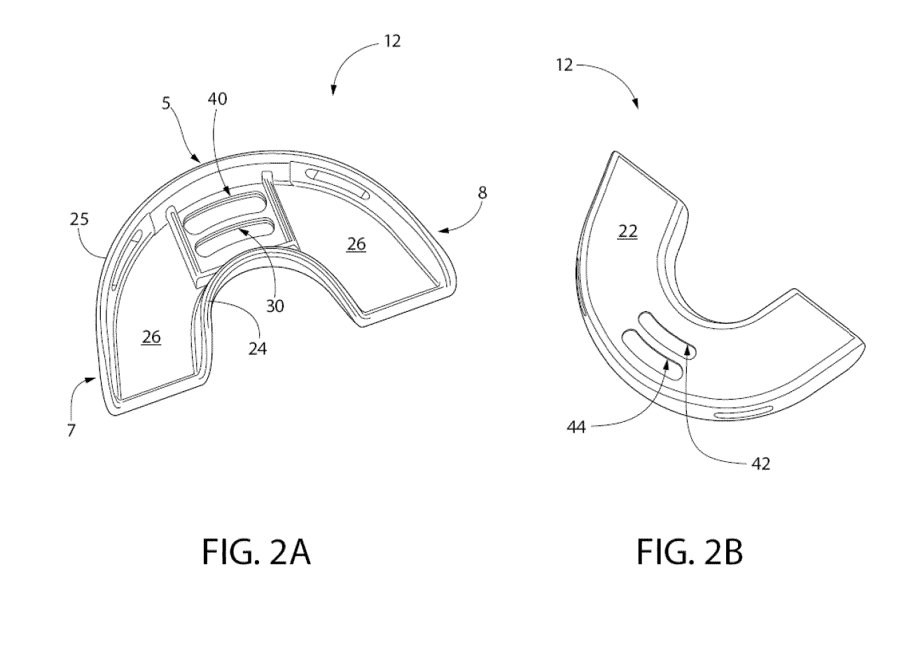

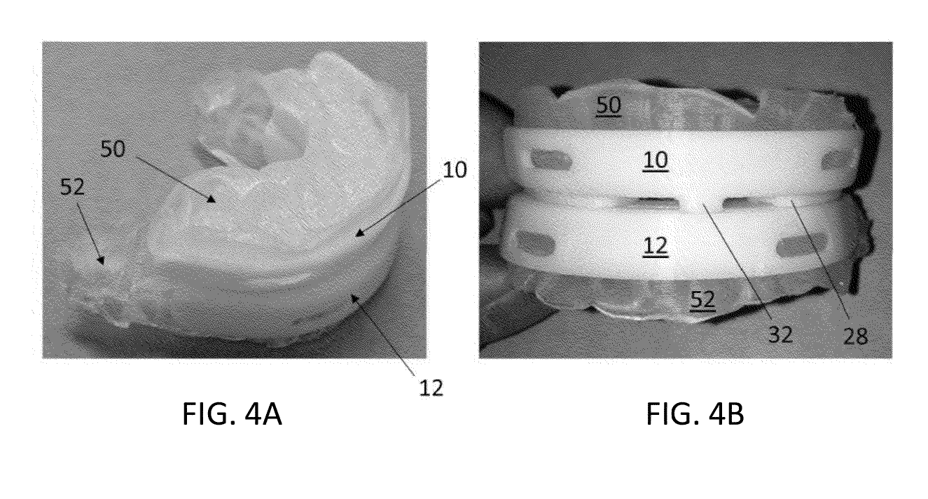

As depicted in FIGS. 1 and 2, top and bottom views are provided of a first exemplary tray component 10 (FIG. 1) and a second exemplary tray component 12 (FIG. 2) of the present invention. It should be appreciated that components 10 and 12 may each be used either as a maxillary tray component or as a mandibular tray component, and desired. Further still, it should be appreciated that there is no limitation as to the pairing of particular first and second tray components when preparing a full assembly of first and second trays with dental inserts, an example of which is shown in FIG. 4. For example, in one embodiment, tray 10 may serve as a first, maxillary tray component, while tray 12 may serve as a second, mandibular tray component, such that components 10 and 12 may each be fitted with a dental insert to form the completed assembly shown in FIG. 4.

Referring again to FIGS. 1 and 2, trays 10 and 12 may include a generally U-shaped body 20 with an arcuate end region 5 aligning with a subject's incisors, and two extending branches 7 and 8 from end region 5 covering the subject's canines and optionally the premolars and molars. Accordingly, body 20 is sized to fit around a full arch or partial arch of a subject's upper or lower teeth. Body 20 includes a generally flat base region 22 with inner and outer sidewalls 24 and 25 extending upward as shown in the top view (FIGS. 1A and 2A) to form a trough region 26 therebetween. Extensions 7 and 8 of body 20 may further include a bite pad 28 on the bottom surface on the first and/or second tray component, for example as shown in FIG. 1B. It should be appreciated that there is no limitation to the geometry or dimensions of the bite pad. As contemplated herein, at least one of the first and second tray components should each have bite pads that, when assembled and positioned in the mouth of a subject, effectively separate the teeth and distribute or offload pressure generated by the jaw muscles.

The trays as described herein may be manufactured via standard molding and/or printing techniques using acrylic or polycarbonate resins, polypropylene or other sturdy polymeric materials suitable for trays and other dental applications as would be understood by those skilled in the art.

In one embodiment, the first and second tray components may include a temporary engagement mechanism 30 for temporarily interlocking the first and second tray components when assembled, thereby controlling the degree of movement of the first and second tray components in relation to each other when assembled in the subject's mouth. For example, engagement mechanism 30 may include the following features. As shown in FIG. 1B, the bottom portion of maxillary tray component 10 may include at arcuate end 5 a block or plate 32 from which a post 34 extends upward to a generally rectangular or oblong shaped flange 36, thereby forming a T-Bar. Mandibular tray component 12 may include at its arcuate end 5 one or more slots or channels 40 running substantially parallel to the bight of end 5. For example, tray 12 may include 1, 2, or even 3 or more slots. As contemplated herein, slots 40 may be substantially straight, or they may be slightly arcuate to follow the curve of arcuate end 5. Further, when two or more slots 40 are used, these slots may be the same length, or they may be of variable length. For example, in one embodiment, the slot nearest to inner sidewall 24 (slot 42) may be of a first, notably shorter length, while additional slots moving outwards towards outer sidewall 25 (such as slot 44) may be of variably increasing lengths. Further, slots 40 may be of a variable width. For example, slot 42 may be generally thinner, such that post 34 of the T-Bar has minimal forward and backward motion when inserted through slot 42, while slot 44 may be slightly wider, such that post 34 of the T-Bar has increased forward and backward motion when inserted through slot 44. In one embodiment, post 34 may be approximately 0.97 inches diameter, and slot 42 may have a width of 0.10 inches, while slot 44 may have a width of 0.14 inches. It should be appreciated that there is no limitation to the specific dimensions of slots 40 or of any portion of the T-Bar.

To engage the bottom portions of the first and second tray components, the length of flange 36 is first aligned with the desired slot 40 and inserted therethrough, such that the entire flange portion 36 passes through the desired slot 40. Next, the first or second tray component is rotated relative to the other so that the length of flange 36 is substantially perpendicular to the length of slot 40 when the arcuate end regions 5 of the first and second tray components are in alignment to fit the upper and lower arches of the subject's teeth, thereby interlocking the components as shown in FIGS. 4 and 7-10. The reverse of these steps are performed to disengage the first and second tray components when removed from the subject's mouth.

Accordingly, when the first and second tray components are engaged and positioned in the subject's mouth, the tray components provide some, but limited, forward and backward movement relative to each other, depending on the width of the slot selected. In a preferred embodiment, a small amount of forward and backward movement is preferred, as this movement allows for freedom of jaw movement during sleep, thereby reducing jaw tension. Furthermore, when the first and second tray components are engaged and positioned in the subject's mouth, limited lateral movement is permitted as post 34 slides within slot 40, based on the length of slots 40. Vertical movement is limited to the length of post 34.

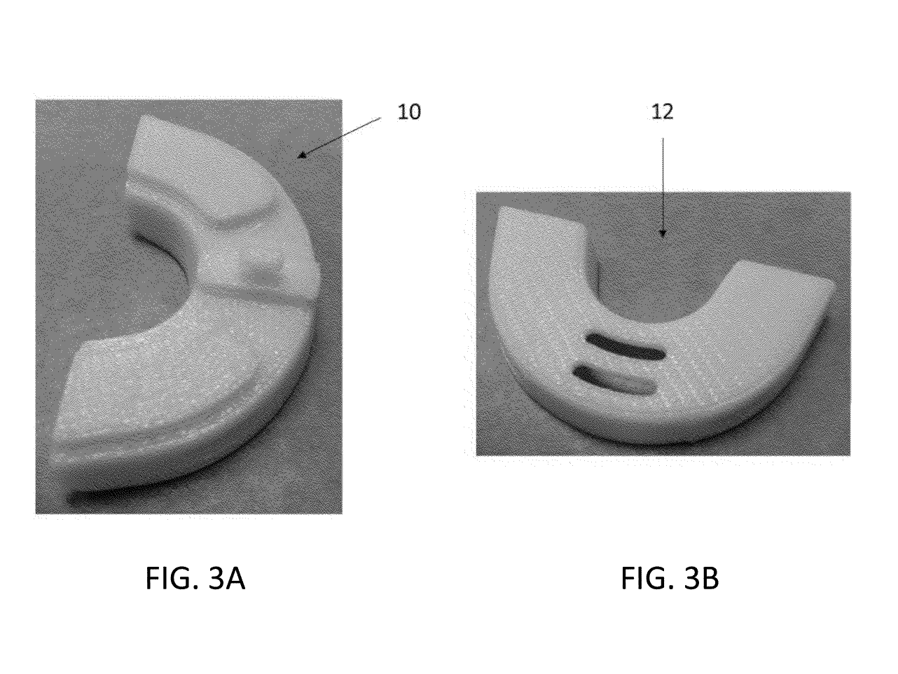

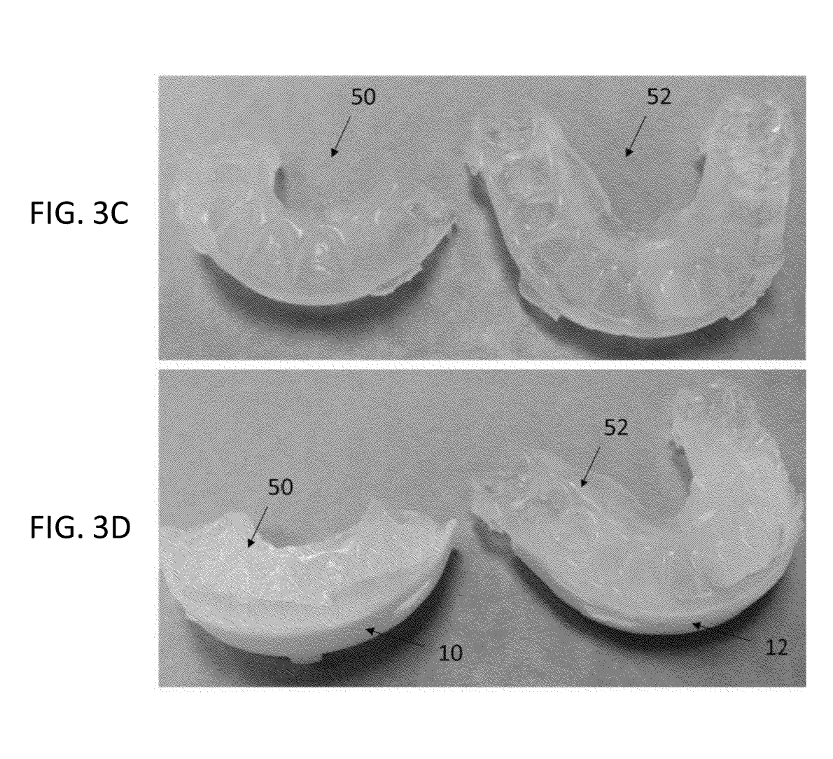

FIG. 3 illustrates the inclusion of dental inserts 50 and 52 molded to a subject's dentition, to improve the comfort of the trays when positioned in the mouth. Dental inserts 50 and 52 as shown in FIG. 3C are shaped to fit into troughs 26 of the trays (FIG. 3D), including tray components 10 and 12 as shown in FIGS. 3A and 3B to form first and second component assemblies. Dental inserts 50 and 52 may be composed of any suitable deformable material, such as thermoplastic molding materials that are deformable with moderate heating and return to a hardened state when cooled to room temperature.

In one embodiment, the dental inserts for the first and second components each substantially cover the full arch of upper and lower teeth. In another embodiment and as shown in FIG. 4A, the present invention includes a first component assembly having the dental insert portion cover the full arch of teeth and a second component assembly having the dental insert portion cover only a partial arch of teeth. For example, in one embodiment, the second component dental insert may cover only the incisors and canines In another embodiment, the second component dental insert may cover only the incisors, canines and first premolars. In another embodiment, the second component dental insert may cover only the incisors, canines and premolars. In another embodiment, the second component dental insert may cover only the incisors, canines, premolars and first molars. In another embodiment, the second component dental insert may cover only the incisors, canines, premolars and first and second molars.

In another embodiment, the first component dental insert may cover only a partial arch of teeth, and the second component dental insert may similarly cover only a partial arch of teeth, where the second component dental insert covers a smaller number of teeth than the first component dental insert. For example, in one embodiment, the first component dental insert may cover all but the third molars, and the second component dental insert may cover all but the second and third molars, or cover the incisors, canines and premolars, or cover the incisors, canines and first premolars, or cover just the incisors and canines. In another embodiment, the first component dental insert may cover all but the second and third molars, and the second component dental insert may cover the incisors, canines and premolars, or cover the incisors, canines and first premolars, or cover just the incisors and canines. In another embodiment, the first component dental insert may cover the incisors, canines and premolars, while the second component dental insert may cover the incisors, canines and first premolars, or cover just the incisors and canines In another embodiment, the first component dental insert may cover the incisors, canines and first premolars, while the second component dental insert may cover just the incisors and canines.

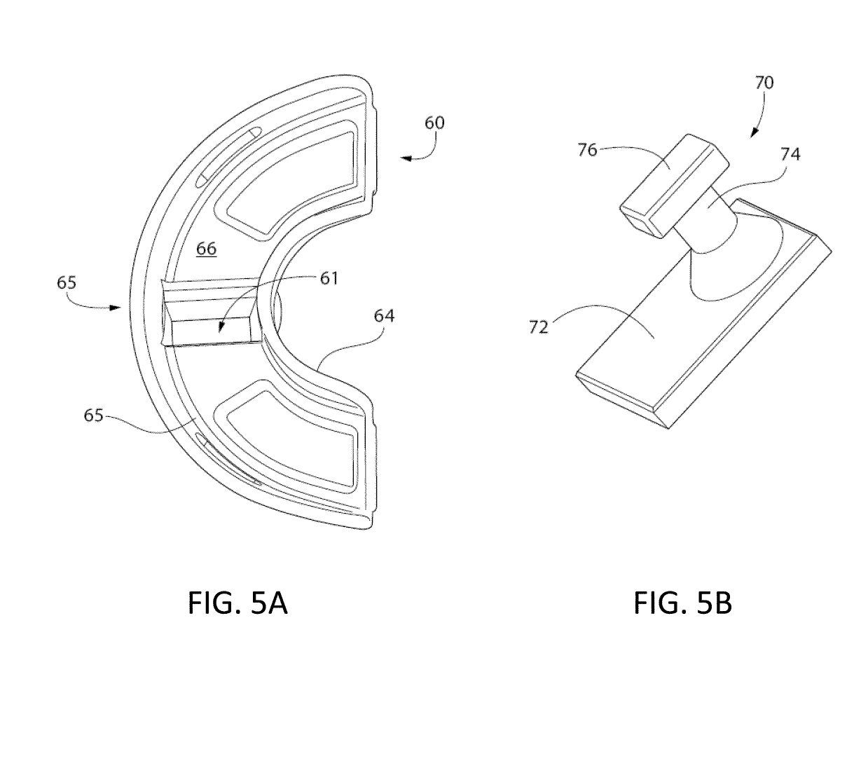

In another embodiment as shown in FIGS. 5-10, The T-Bar may be removable from the tray component. For example, as shown in FIG. 5A, the tray component 60 includes at its arcuate end region an opening 61 within the trough region 66 formed between inner and outer sidewalls 64 and 65. Removable T-Bar 70 includes a base plate 72, a post 74 extending from base plate 72 and a generally rectangular or oblong shaped flange 76 at the extended end of post 74. In one embodiment, post 74 is positioned off center of base plate 72, such as near one side of base plate 72 as shown in FIG. 5B. To assemble the tray component, T-Bar 70 is inserted within opening 61 such that it is at least temporarily affixed to tray component 60 via a friction fit, a detent or other temporary attachment or engagement mechanism.

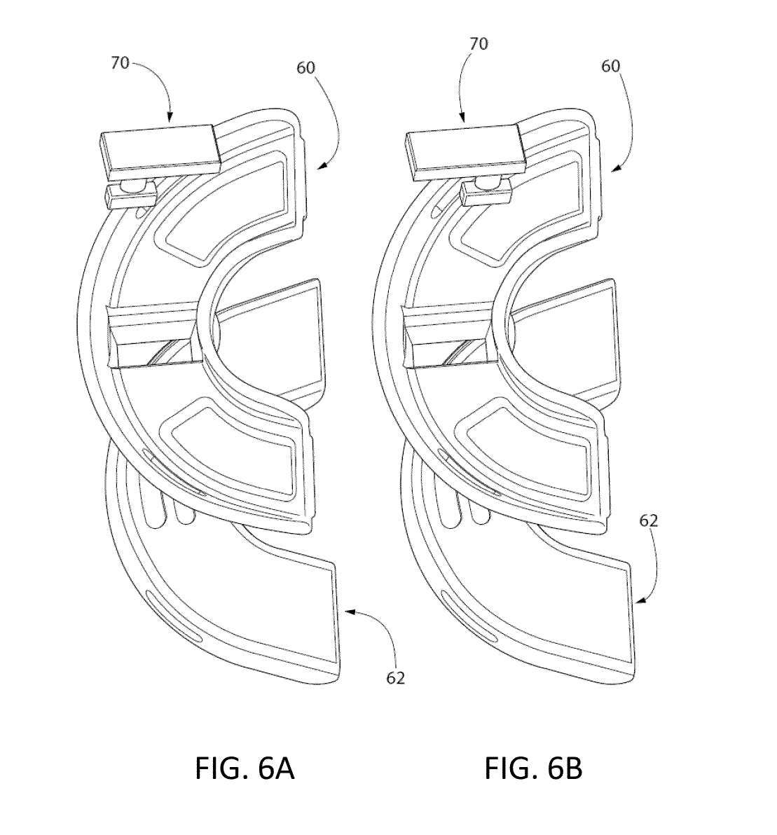

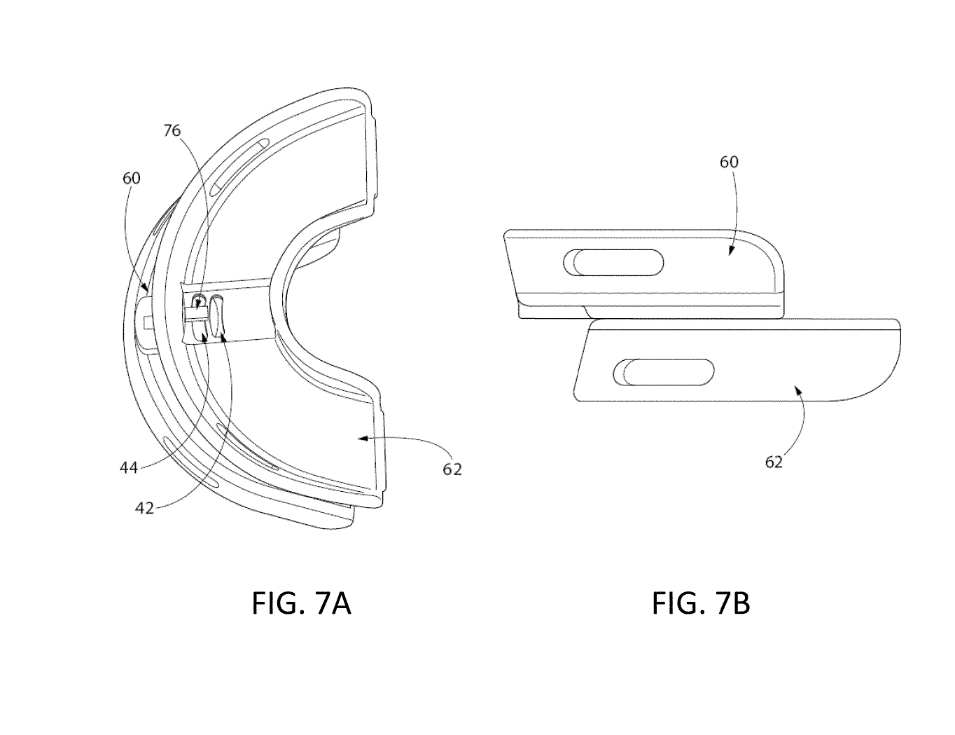

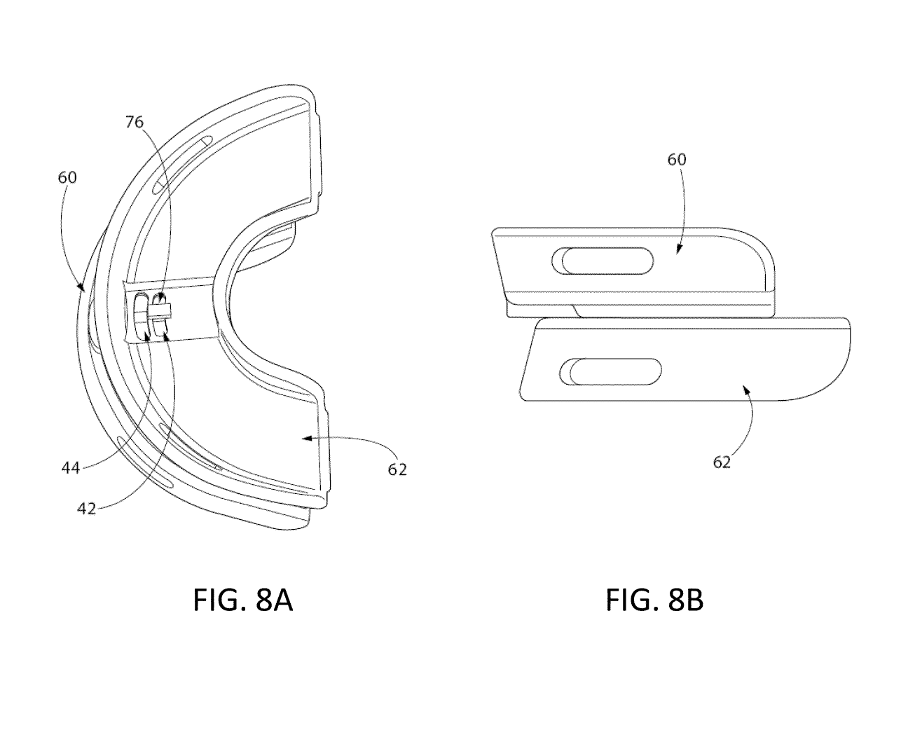

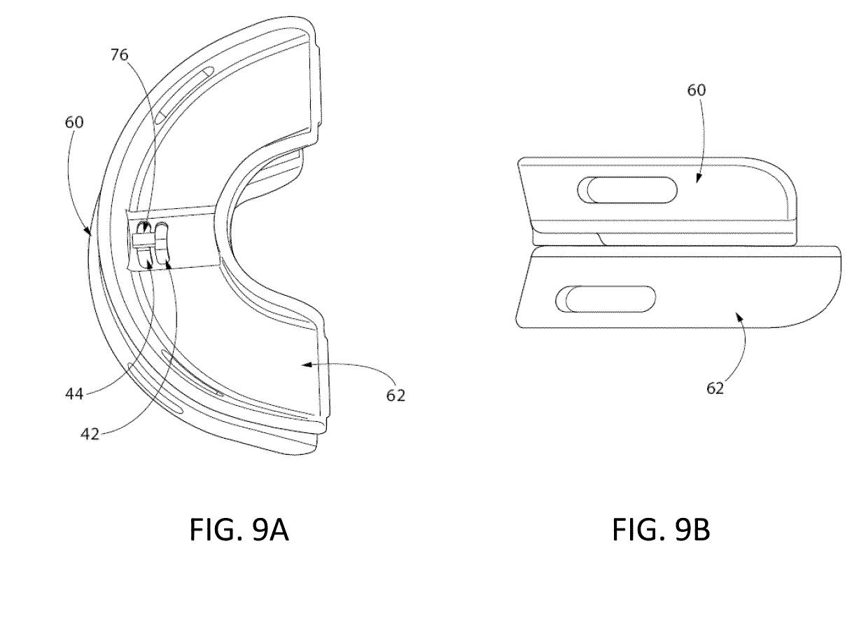

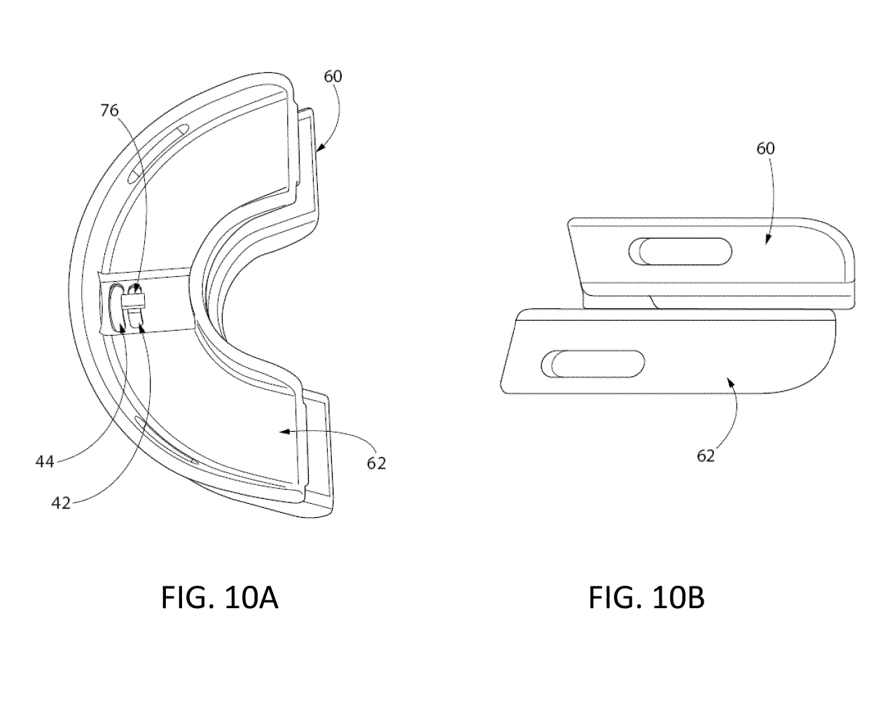

Because post 74 is located to one side of base plate 72, the final position of post 74 and flange 76 can be one of two options. For example, as shown in FIG. 6A, post 74 and flange 76 may be in a more forward position, or as shown in FIG. 6B, post 74 and flange 76 may be positioned further back. These two positioning options for T-Bar 70, when used in conjunction with multiple slots 40 of the second tray component, can create four or more configurations of the first tray relative to the second tray when interlocked. For example, as shown in FIGS. 7A and 7B, the bruxism guard may be interlocked in a first position, where T-Bar 70 is positioned in component 60 with post 74 in the back position, and T-Bar 70 interlocks with component 62 in slot 44 near the outer edge. In this first position, the subject's mandible is largely retracted. As shown in FIGS. 8A and 8B, the bruxism guard may be interlocked in a second position, where T-Bar 70 is positioned in component 60 with post 74 in the back position, and T-Bar 70 interlocks with component 62 in slot 42 near the inner edge. In this second position, the subject's mandible is slightly retracted. As shown in FIGS. 9A and 9B, the bruxism guard may be interlocked in a third position, where T-Bar 70 is positioned in component 60 with post 74 in the forward position, and T-Bar 70 interlocks with component 62 in slot 44 near the outer edge. In this third position, the subject's mandible is slightly protracted. Lastly, as shown in FIGS. 10A and 10B, the bruxism guard may be interlocked in a fourth position, where T-Bar 70 is positioned in component 60 with post 74 in the forward position, and T-Bar 70 interlocks with component 62 in slot 42 near the inner edge. In this fourth position, the subject's mandible is largely protracted. It should be appreciated that the total number of final configurations is determined by the offset positioning of the T-Bar inserted into the first tray component, and the number of slots in the second tray component.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A bruxism guard comprising:
a first U shaped tray having a top surface and a bottom surface, the top surface including a trough with a dental insert positioned therein, wherein the dental insert substantially conforms to a full arch of a subject's teeth; and
a second U shaped tray having a top surface and a bottom surface, the top surface including a trough with a dental insert positioned therein, wherein the dental insert substantially conforms to a only a partial arch of the subject's teeth;
wherein the bottom surfaces of the first and second trays oppose each other when positioned within the subject's mouth.

2. The bruxism guard of claim 1, wherein the dental insert of the first tray conforms to the subject's mandibular dentition and the dental insert of the second tray conforms to the subject's maxillary dentition.

3. The bruxism guard of claim 1, further comprising an interlocking mechanism to temporarily secure the first tray to the second tray.

4. The bruxism guard of claim 3, wherein the interlocking mechanism permits limited forward and backward movement between the first and second trays.

5. The bruxism guard of claim 3, wherein the interlocking mechanism permits lateral movement between the first and second trays.

6. The bruxism guard of claim 3, wherein the bottom surface of the first component includes a plurality of slots at its arcuate end, and the bottom surface of the second component includes a T-Bar sized to temporarily engage any one of the plurality of slots in the first component.

7. The bruxism guard of claim 6, wherein the slot nearest the far edge of the arcuate end of the first component is wider than a second adjacent slot.

8. The bruxism guard of claim 1, wherein at least one of the first or second trays includes a bite plate on the bottom surface of the tray.

9. The bruxism guard of claim 1, wherein the dental insert of the second tray cover's the only the incisors and canines of the subject's arch of teeth.

10. The bruxism guard of claim 1, wherein the dental insert of the second tray cover's the only the incisors, canines and first premolars of the subject's arch of teeth.

11. The bruxism guard of claim 1, wherein the dental insert of the second tray cover's the only the incisors, canines and premolars of the subject's arch of teeth.

12. The bruxism guard of claim 1, wherein the dental insert of the second tray cover's the only the incisors, canines, premolars and first molars of the subject's arch of teeth.

13. The bruxism guard of claim 1, wherein the dental insert of the second tray cover's the only the incisors, canines, premolars and first and second molars of the subject's arch of teeth.

14. A bruxism guard comprising:
a first U shaped tray having a top surface and a bottom surface, the top surface including a trough with an opening positioned therein;
a T-Bar having a base plate, a post extending off center from the base plate and a flange at the extended end of the post; and
a second U shaped tray having a top surface and a bottom surface, the top surface including a trough with at least one slot creating an opening therethrough;
wherein when the T-Bar is positioned in the opening of the first tray with the T-Bar post and flange extending therethrough, the T-Bar temporarily engages one of the at least one slots to interlock the first tray to the second tray.

15. The bruxism guard of claim 14, wherein the first tray and the second tray are capable of interlocking in at least two different configurations for each slot within the second tray.

16. The bruxism guard of claim 14, further comprising a dental insert positioned within the trough of each of the first tray and the second tray.

17. The bruxism guard of claim 16, wherein at least one of the dental inserts substantially conforms to a full arch of the subject's teeth.

18. The bruxism guard of claim 16, wherein at least one of the dental inserts substantially conforms to a partial arch of the subject's teeth.

19. The bruxism guard of claim 16, wherein one dental insert substantially conforms to a partial arch of the subject's teeth and the second dental insert substantially conforms to a full arch of the subject's teeth.

20. The bruxism guard of claim 14, wherein slight forward and backward movement between the first and second trays is permitted when the T-Bar engages the at least one slot.

21. The bruxism guard of claim 14, wherein lateral movement between the first and second trays is permitted when the T-Bar engages the at least one slot.

22. The bruxism guard of claim 20, wherein the second tray includes two or more slots, and the width of the first slot is greater than the width of the second slot.
